(19) United States
(12) Patent Application Publication
(10) Pub. No.: US 2003/0152655 A1
(43) Pub. Date: Aug. 14, 2003

(54) Method and composition for promoting hair growth

(76) Inventor: Burkhard Grallert, Douglas (GB)

Correspondence Address: Melvin I Stoltz, 51 Cherry Street, Milford, CT, 06460, US

(21) Appl. No.: 10/221,243

(22) PCT Filed: Feb. 9, 2001
(86) PCT No.: PCT/GB01/00546

(30) Foreign Application Priority Data
Feb. 9, 2000 (GB) 0002836.5

(51) Int. Cl. A61K 35/78

(52) U.S. Cl. 424/757; 424/725

(57) ABSTRACT

A composition for preventing hair loss and promoting hair growth comprises fenugreek extract and a carrier, for example, hydroxylated lecithin and glycerine. The composition is preferably in the form of a spray which is applied beneath the tongue for translingual absorption.

### FIELD OF THE INVENTION

This invention relates to a method and composition for promoting hair growth.

### SUMMARY OF THE INVENTION

According to a first aspect of the present invention there is provided a method of promoting hair growth which includes the use of a formulation containing fenugreek extract.

The formulation is preferably applied topically so as to be absorbed trans-lingually into the bloodstream.

The formulation is preferably applied in the form of a spray.

The fenugreek extract is preferably mixed with a carrier, a vasodilator, a stabilising agent, a DHT inhibitor, a buffering agent and a sweetener.

The carrier may comprise hydroxylated lecithin and glycerine. The vasodilator may be L-Arginine. The DHT inhibitor is preferably a zinc salt such as zinc gluconate. The buffering agent is preferably citric acid and the sweetener is preferably stevia extract.

The formulation may alternatively be in the form of a capsule which is ingested. Trans-lingual absorption of the fenugreek extract is, however, the more effective mode of application.

According to a second aspect of the present invention there is provided a method of preparing a composition for promoting hair growth, which method includes mixing fenugreek extract with a carrier.

The fenugreek extract is preferably contained within a lipospray formulation which includes hydroxylated lecithin and glycerine for promoting trans-lingual absorption of the fenugreek extract into the bloodstream.

According to a third aspect of the present invention there is provided a composition for promoting hair growth, said composition including fenugreek extract and a lipospray formulation which includes hydroxylated lecithin and glycerine for promoting trans-lingual absorption of the fenugreek extract into the bloodstream.

The composition may contain from 1 to 20% by weight of fenugreek extract.

The composition preferably also includes a sweetening agent to counter the bitter taste of the fenugreek extract. A preferred sweetening agent is stevia extract. The amount by weight of stevia extract in the composition is preferably within the range of from 5 to 15% of the weight of the fenugreek extract.

The composition may also include a natural flavour, for example, raspberry flavour, to make it more palatable. A buffering agent, for example, citric acid, may also be included to maintain the pH of the composition within a desired range. The composition may also include a stabilising agent, for example, potassium sorbate.

The composition may additionally or alternatively include folic acid, vitamins B2, B6 and B12. It may also include ginseng, ginko biloba and/or calcium pantotenate.

### DESCRIPTION OF THE PREFERRED EMBODIMENTS

A lipospray formulation is prepared containing the following ingredients:

a) distilled water,

b) glycerine,

c) hydroxylated lecithin,

d) natural raspberry flavour,

e) fenugreek extract (Trigonella foenum gracum, seed),

f) stevia extract (Stevia rebandiana, leaves, 80% Steviosides,

g) potassium sorbate, and

h) citric acid.

On preparing 0.7 ml. of the above composition for application by means of a spray, 20 mg. of fenugreek extract was employed. The amount of stevia extract which was employed was 1.5 mg. For that volume of the composition, the amount by weight of fenugreek extract may be varied from about 7 mg. to about 140 mg. The stevia extract is added as a sweetening agent to counter the bitter taste of the fenugreek, the amount by weight of stevia extract being about 5% to about 15% of the weight of the stevia extract. The addition of the natural raspberry flavour to the composition assists in making the composition palatable.

The hydroxylated lecithin and glycerine serve as a carrier to assist trans-lingual absorption of the fenugreek extract into the bloodstream so that, when the user sprays a small quantity of the composition under the tongue, i.e. the composition is applied topically and sub-lingually, the fenugreek extract will be absorbed trans-lingually into the bloodstream.

In addition to the specific ingredients set out above, the composition may alternatively or additionally include one or more of the following:

a) folic acid,

b) vitamin B2,

c) vitamin B6,

d) vitamin B12,

e) calcium pantotenate,

f) ginseng, and

g) ginko biloba.

Regular topical sub-lingual application of the above composition has been found to prevent hair loss and promote the growth of hair.

An alternative composition for use in capsule form for ingestion includes the following ingredients:

a) distilled water,

b) glycerine,

c) polysorbate 20,

d) fenugreek extract,

e) L-Arginine,

f) hydroxylated lecithin,

g) propylene glycol,

h) citric acid,

i) raspberry flavour,

j) niacinamide,

k) stevia extract,

l) potassium sorbate,

m) pentathenic acid,

n) zinc gluconate, and

o) vitamins.

On preparing 0.7 ml. of the above composition, 20 mg. of fenugreek extract was employed, together with 15.5 mg. of L-Arginine, 3.0 mg. of stevia extract (i.e. Stevia rebandiana, leaf, 80% Steviosides), 5.8 mg. of niacinamide, 1.0 mg. of zinc gluconate and up to 10 mg. of vitamins and pentathenic acid.

Taking the above composition regularly in capsule form, with each capsule containing 0.7 ml. of the formulation has also been found to prevent hair loss and promote the growth of hair. Clinical trials have shown that, when one capsule of the above composition was taken daily for a period of six months by a group of 43 men who had previously suffered hair loss, the following results were obtained:

a) a slight increase—7 men,

b) a moderate increase—28 men, and

c) a clear increase—8 men.

There was no significant variation in the degree of improvement which was obtained for different age groups.

1. A method of promoting hair growth which includes the use of a formulation containing fenugreek extract.

2. A method as claimed in claim 1, in which the formulation is applied topically so as to be absorbed trans-lingually into the bloodstream.

3. A method as claimed in claim 2, in which the formulation is applied in the form of a spray.

4. A method of preparing a composition for promoting hair growth, which method includes mixing fenugreek extract with a carrier.

5. A method as claimed in claim 4, which includes mixing a vasodilator with the fenugreek extract and the carrier.

6. A method as claimed in claim 4, in which the fenugreek extract is contained within a lipospray formulation which includes hydroxylated lecithin and glycerine for promoting trans-lingual absorption of the fenugreek extract into the bloodstream of the user.

7. A composition for promoting hair growth, said composition including fenugreek extract and a lipospray formulation which includes hydroxylated lecithin and glycerine.

8. A composition as claimed in claim 7, which contains from 1 to 20% by weight fenugreek extract.

9. A composition as claimed in claim 8, which also includes a sweetening agent to counter the bitter taste of the fenugreek extract.

10. A composition as claimed in claim 9, in which the sweetening agent is stevia extract.

11. A composition as claimed in claim 10, in which the amount by weight of stevia extract in the composition is within the range of from 5 to 15% of the weight of the fenugreek extract.

12. A composition as claimed in claim 7, which includes a buffering agent.

13. A composition as claimed in claim 12, in which the buffering agent is citric acid.

14. A composition as claimed in claim 7, which includes a stabilising agent.

15. A composition as claimed in claim 14, in which the stabilising agent is potassium sorbate.
